(19) United States
(12) Patent Application Publication
(10) Pub. No.: US 2026/0308128 A1
(43) Pub. Date: Oct. 8, 2026

(54) BETA-ELEMENE DERIVATIVE CONTAINING PHOTOAFFINITY GROUP DIAZIRINE, AND PREPARATION METHOD AND USE THEREOF AS PHOTOAFFINITY MOLECULAR PROBE

(71) Applicant: HANGZHOU NORMAL UNIVERSITY, Hangzhou (CN)

(72) Inventors: Tian Xie, Hangzhou (CN); Xiangyang Ye, Hangzhou (CN); Yingqian Zhang, Hangzhou (CN); Qing Mao, Hangzhou (CN); Chenyuan Wu, Hangzhou (CN); Renren Bai, Hangzhou (CN)

(21) Appl. No.: 18/996,773

(22) PCT Filed: Jun. 28, 2023
(86) PCT No.: PCT/CN2023/102980

§ 371 Date: Jan. 17, 2025

(30) Foreign Application Priority Data
Sep. 30, 2022 (CN) 202211212049.4

(51) Int. Cl. C07D 229/02; A61P 35/00; C07D 487/04; G01N 33/50

(52) U.S. Cl. CPC C07D 229/02; A61P 35/00; C07D 487/04; G01N 33/5011

(57) ABSTRACT

A β-elemene derivative containing a photoaffinity group diazirine, and a preparation method and use thereof as a photoaffinity molecular probe are provided. A compound having a structure shown in Formula (I), a pharmaceutical composition containing the compound having the structure shown in Formula (I), and a hydrate thereof, as well as an isotopic derivative, a chiral isomer, an allomer, different salts, prodrugs, and preparations of these compounds are provided. The preparation method and the use of the β-elemene derivative containing the photoaffinity group diazirine are also provided, where the compound has an anti-proliferation activity on various tumor cell lines, and is applied to screening of a β-elemene interacting protein. The β-elemene photoaffinity molecular probe is expected to become a molecular tool for targeted screening and action mechanism research of an elemene.

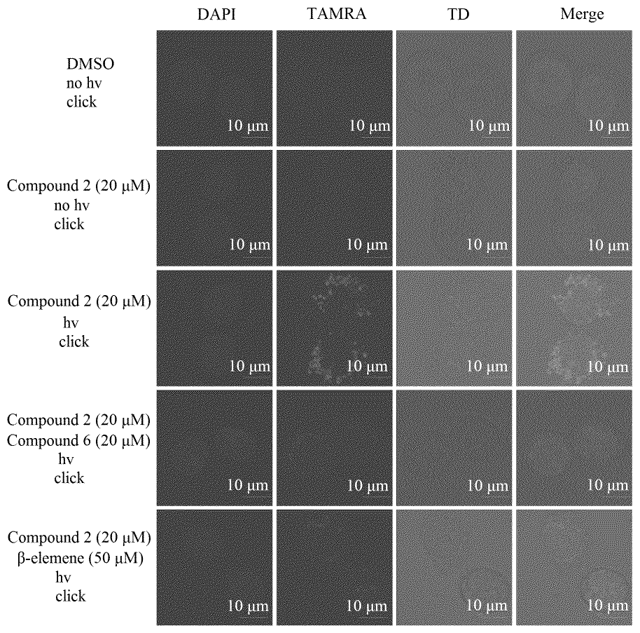

### CROSS REFERENCE TO RELATED APPLICATION

The present application is a national stage application of International Patent Application No. PCT/CN2023/102980, filed on Jun. 28, 2023, which claims priority to Chinese Patent Application No. CN202211212049.4 filed with the China National Intellectual Property Administration (CNIPA) on Sep. 30, 2022 and entitled “BETA-ELEMENE DERIVATIVE CONTAINING PHOTOAFFINITY GROUP DIAZIRINE, AND PREPARATION METHOD AND USE THEREOF AS PHOTOAFFINITY MOLECULAR PROBE”. The disclosure of the two applications is incorporated herein by reference in its entirety.

### TECHNICAL FIELD

The present disclosure belongs to the technical field of preparation and application of photoaffinity molecular probes, and specifically relates to a β-elemene derivative containing a photoaffinity group diazirine, and a preparation method and use thereof as a photoaffinity molecular probe.

### BACKGROUND

Elemene, as a sesquiterpenoid anticancer active ingredient extracted from the tuber of Curcuma wenyujin, one of the “Eight Herbs of Zhejiang Province”, is clinically safe and effective in the treatment of lung cancer, liver cancer, and brain glioma, and has the advantages of sensitizing and attenuating to targeted and chemotherapeutic drugs. In 1994, the State Food and Drug Administration approved elemene as an anti-tumor herbal medicine with independent intellectual property rights in China.

A key to the development of novel drugs is to deeply understand the mechanism of action of drugs in vivo and select the best achievable target for drug efficacy. It is generally believed that elemene can exert anti-tumor effects by intervening in multiple signal transduction pathways, such as inhibiting tumor cell proliferation, inducing apoptosis of cancer cells, inhibiting tumor angiogenesis, inhibiting tumor cell invasion and metastasis, and regulating immune function. However, many studies on elemene remain on the observation of changes in its biological manifestations, and its specific molecular mechanisms and targets remain unclear.

Chemical molecular probes are a type of functionalized molecular tool that can bind to relevant research objects through covalent or non-covalent bonds and be monitored by means of radioactivity, fluorescence, and chemical reactions, thereby obtaining information such as the location and action target of important biological macromolecules in cells. The design of chemical small molecule probes is based on sufficient structure-activity relationship research, and various functional modules are introduced at appropriate positions to achieve different detection purposes without affecting the activity of the compound. General active molecular probes are composed of two parts: one part is an active small molecule containing a reactive group, which can interact with the target; the other part is a reporter group, which is used for detecting and separating the biological target bound by the active small probe, including a fluorophore (for visualization research of biological targets), biotin (for enrichment and purification of target molecules), or a bioorthogonal reaction functional group (to avoid the influence of a labeling group on the interaction between the active small molecule and the biological target), such as an alkyne group or an azide. For active small molecules that are inert in chemical reactions and interact with targets through non-covalent force, in order to reduce the false positive caused by non-specific adsorption, an additional photoaffinity group needs to be introduced in the design of molecular probes. Highly active intermediates such as carbene are generated by light irradiation, and undergo covalent cross-linking with the target, thereby increasing the scope of target screening.

Many well-known natural products have been successfully molecularly probed and used in the study of their corresponding molecular mechanisms of action, action targets, and signaling pathways. For example, in order to explore the mechanism of action of a natural product with strong cytotoxicity, i.e., peptide alkaloid chlorinated cyclic peptide A, Harran's research group has synthesized biotin-labeled chlorinated cyclic peptide and applied it to find that its direct action target is ornithine aminotransferase. Ainsliatrimer, a natural product, is a trimer of guaianolide, a secondary metabolite of the Asteraceae plant Ainsliaea glabra, and shows strong cytotoxicity to a variety of tumor cells, but its specific mechanism of action is still unclear. Lei Xiaoguang's research group combined the total synthesis of a natural product with bioorthogonality, and revealed for the first time that peroxisome proliferator-activated receptor (PPAR) is a direct action target of the natural product using natural product molecular probing.

Elemene is a sesquiterpene compound containing only carbon and hydrogen elements. It is speculated that the interaction of elemene with a target molecule is a non-covalent binding with a weak binding force. Therefore, it is necessary to develop a method for constructing elemene photoaffinity molecular probes for the screening of an action target of elemene in vivo, the exploration of a structure-activity relationship of elemene, and the study of a mechanism of action of elemene.

### SUMMARY

In view of the deficiencies in the prior art, a first object of the present disclosure is to provide a β-elemene derivative containing a photoaffinity group diazirine.

The present disclosure provides a β-elemene derivative containing a photoaffinity group diazirine, or an optical isomer, a racemate, a single enantiomer, and a possible diastereomer thereof, or a pharmaceutically acceptable salt, a prodrug, a deuterated derivative, a hydrate, and a solvate thereof, where the β-elemene derivative containing the photoaffinity group diazirine has a structure shown in Formula (I):

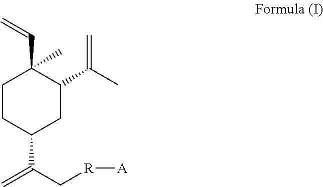

where

A represents

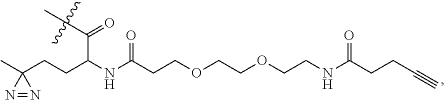 [SMILES: *C(=O)C(CCC1(C)N=N1)NC(=O)CCOCCOCCNC(=O)CCC#C]

Where “ [symbol] ” represents a site where A is linked to R; and

R is dependently selected from the group consisting of the following structural fragments:

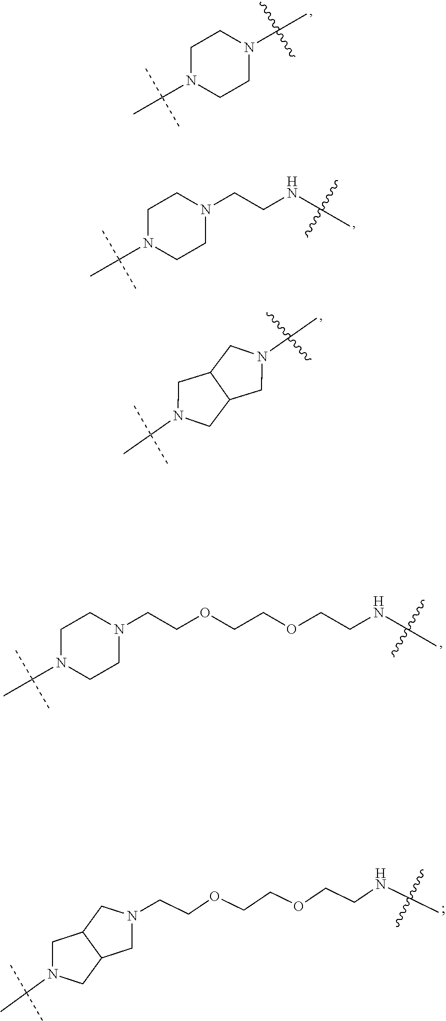

where “ [symbol] ” represents a site where R is linked to elemene, and “ [symbol] ” represents a site where R is linked to A.

In some embodiments, the β-elemene derivative containing the photoaffinity group diazirine has the structure being any one selected from the group consisting of:

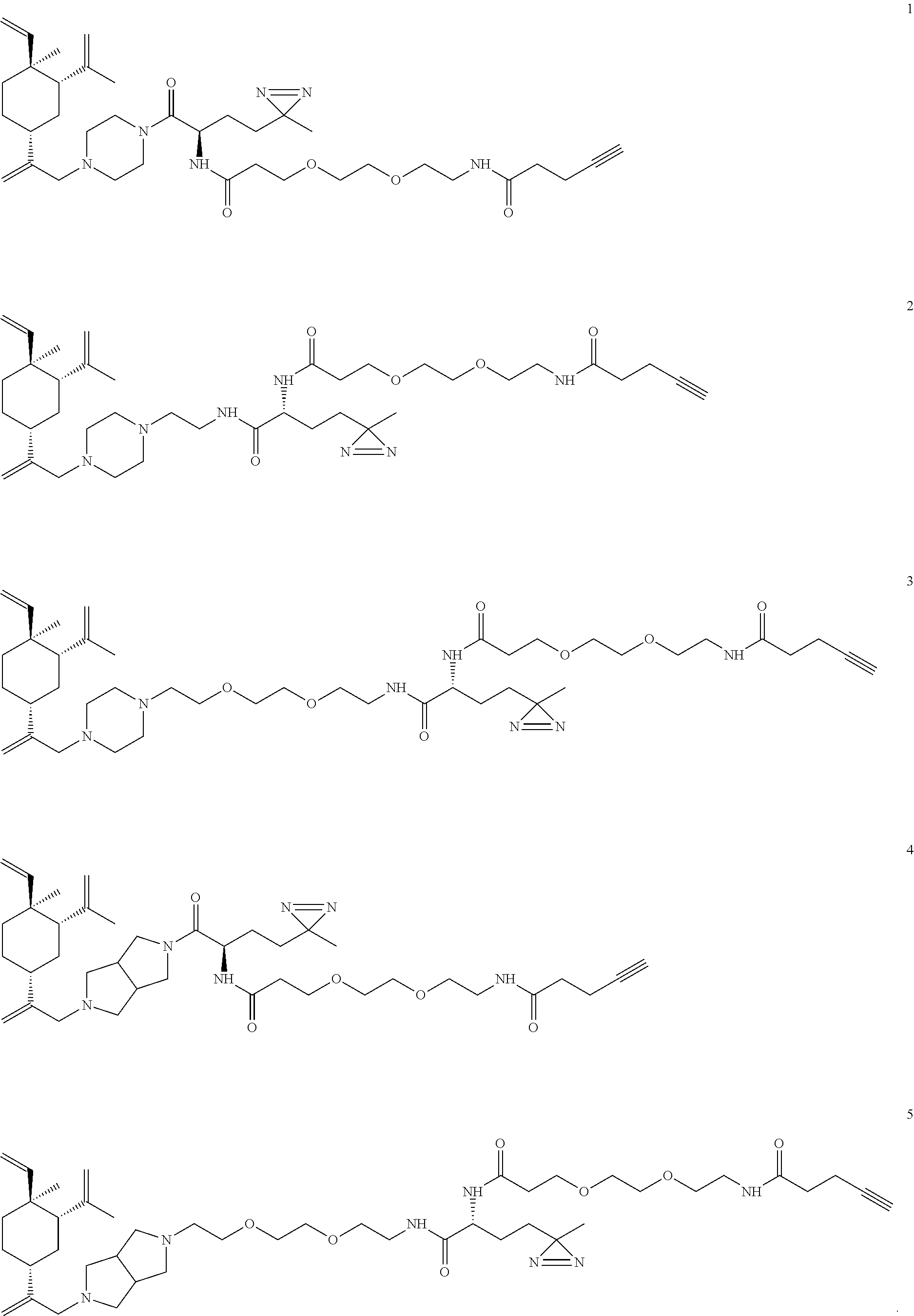

A second object of the present disclosure is to provide a method for preparing the β-elemene derivative containing the photoaffinity group diazirine, including:
(1) subjecting a β-elemene (A-1) to bromine substitution at a 13-allyl position to obtain an intermediate A-2;
(2) linking an R structural fragment A-3 containing a nitrogen heteroatom functional group to the intermediate A-2 through selective nucleophilic substitution to obtain an intermediate A-4;
(3) subjecting a structural fragment at a 13-β-elemene position of the intermediate A-4 to deprotection to obtain an intermediate A-5; and
(4) subjecting the intermediate A-5 and a probe fragment A-OH to intermolecular amide condensation to obtain the derivative having a structure shown in Formula (I);
where a synthetic path is as follows:

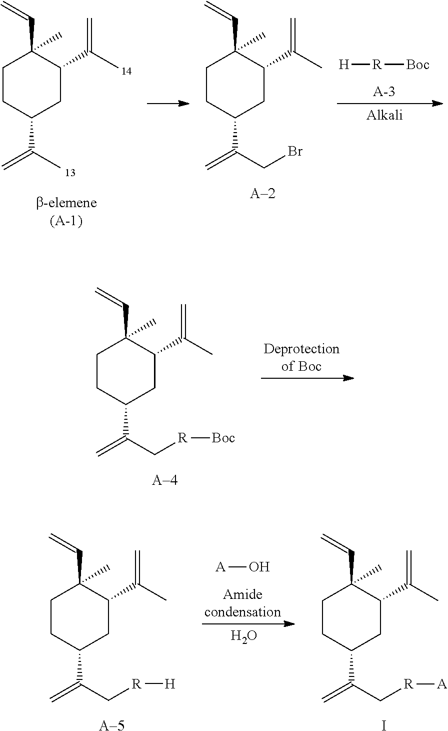

In the present disclosure, the compound shown in Formula (I) can be prepared by the above method; however, the conditions for the method, such as reactants, solvents, amounts of the compounds used, reaction temperature, and reaction time are not limited to the above explanations. The compound can also be conveniently prepared by optionally combining various synthetic methods described in this description or known in the art. Such combinations can be easily conducted by those skilled in the art to which the present disclosure relates.

A third object of the present disclosure is to provide use of the β-elemene derivative containing the photoaffinity group diazirine, or the optical isomer, the racemate, the single enantiomer, and the possible diastereomer thereof, or the pharmaceutically acceptable salt, the prodrug, the deuterated derivative, the hydrate, and the solvate thereof as a β-elemene photoaffinity molecular probe.

A fourth object of the present disclosure is to provide a method for screening a β-elemene interacting protein, including using the β-elemene derivative containing the photoaffinity group diazirine, or the optical isomer, the racemate, the single enantiomer, and the possible diastereomer thereof, or the pharmaceutically acceptable salt, the prodrug, the deuterated derivative, the hydrate, and the solvate thereof as a photoaffinity molecular probe.

Some embodiments of the present disclosure have the following advantages:

In the present disclosure, after introducing a photoaffinity group (diazirine) and a reporter group (alkynyl) onto a β-elemene skeleton and increasing polar and water-soluble groups, a β-elemene derivative with a photoaffinity activity is prepared, and a series of β-elemene photoaffinity molecular probes are synthesized. The β-elemene photoaffinity molecular probe has the activity of inhibiting the proliferation of various tumor cell lines, and can covalently bind to a protein in a cytoplasm after irradiation at a wavelength of 365 nm. The probe can be used to screen a β-elemene interacting protein and is expected to be used to explore the molecular mechanism of action of an elemene.

### BRIEF DESCRIPTION OF THE DRAWINGS

FIGURE shows the cell imaging results of the β-elemene photoaffinity probe (Compound 2) versus the control Compound 6 and β-elemene in Example 10.

### DETAILED DESCRIPTION OF THE EMBODIMENTS

The present disclosure will be further described below with reference to specific examples, but the scope of the present disclosure is not limited thereto.

### Example 1: Preparation of a Compound 1

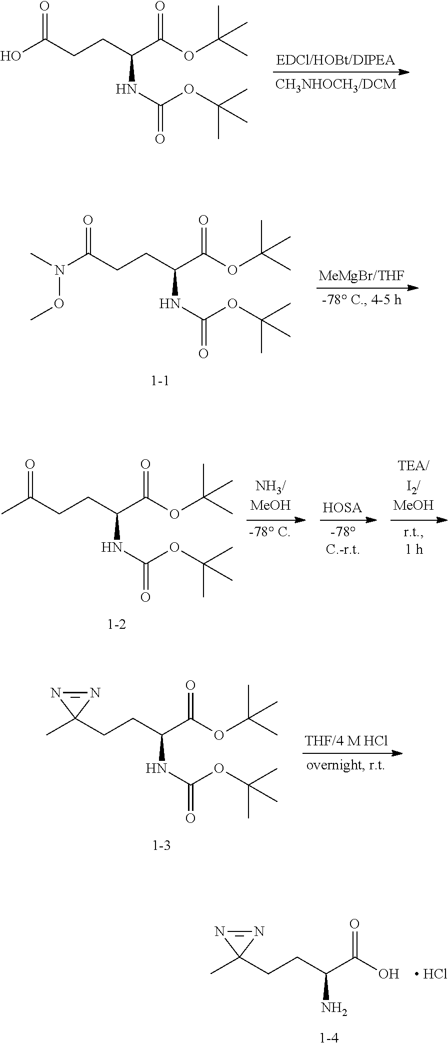

1-(3-dimethylaminopropyl)-3-ethylcarbodiimide hydrochloride (EDCl) (164.5 mg, 0.86 mmol), 1-hydroxybenzotriazole (HOBt) (57.9 mg, 0.43 mmol) and N,N-diisopropylethylamine (DIPEA) (127.7 mg, 0.99 mmol) were added sequentially to a solution of a compound Boc-L-glutamic acid-1-tert-butyl ester (Boc-Glu-OtBu) (100 mg, 0.33 mmol) in DCM at room temperature in an ice bath. A resulting reaction solution was stirred in the ice bath for 1 h, and then methoxymethylamine (41.8 mg, 0.43 mmol) was added thereto. A resulting reaction solution was heated to room temperature, and subjected to reaction for 7 h. A resulting system was subjected to reduced pressure to remove the DCM, and a resulting residue was extracted with ethyl acetate (3×5 mL). A combined organic phase was washed with water (2×5 mL) and saturated brine (2-5 mL) in sequence, dried over anhydrous sodium sulfate, and filtered to remove the desiccant. A resulting filtrate was concentrated under reduced pressure. A resulting crude product was purified by silica gel column chromatography (ethyl acetate/petroleum ether system) to obtain a white solid, Compound 1-1 (103.5 mg, yield 90.7%). ¹H NMR (500 MHz, CDC₃) δ5.19 (d, J=8.2 Hz, 1H), 4.16-4.21 (m, 1H), 3.67 (s, 3H), 3.17 (s, 3H), 2.43-2.59 (m, 2H), 2.10-2.19 (m, 1H), 1.87-1.96 (m, 1H), 1.45 (s, 9H), 1.43 (s, 9H).

A 1 mol/L MeMgBr solution (4.68 mL, 4.68 mmol) was slowly added dropwise into a solution of the Compound 1-1 (360 mg, 1.04 mmol) in THF (7.5 mL) at −78° C. A resulting reaction solution was heated slowly to room temperature and reacted for 4 h. An NH₄Cl solution was added thereto to quench the reaction. A resulting product was subjected to reduced pressure to remove THF, and then subjected to post-treatment in the same manner as for Compound 1-1 to obtain a white solid, Compound 1-2 (253.5 mg, yield 80.9%). ¹HNMR (500 MHz, CDCl₃) δ5.06 (s, 1H), 4.09 (s, 1H), 2.41-2.57 (m, 2H), 2.11 (s, 3H), 2.05-2.06 (m, 1H), 1.72-1.84 (m, 1H), 1.42 (s, 9H), 1.40 (s, 9H).

The Compound 1-2 (83.3 mg, 0.27 mmol) was dissolved in a 7 mol/L NH₃/MeOH solution (1.6 mL, 10.8 mmol) at −78° C. and stirred for 7 h to 8 h. Hydroxylamine sulfonic acid (38.4 mg, 0.34 mmol) was added thereto at −78° C., and a resulting reaction solution was slowly heated to room temperature and subjected to reaction overnight, with ammonia volatilizing at the same time. A resulting reaction product was filtered to remove a white solid, and a resulting residue was washed with methanol (5 mL×3). A resulting mother liquor was concentrated to half its volume. Triethylamine (TEA) and 12 were added to the mother liquor sequentially under an ice bath until a resulting reaction liquid turned brown, and the reaction liquid was heated to room temperature and stirred for 1 h until it turned colorless. A resulting product was subjected to reduced pressure to remove methanol and then subjected to post-treatment in the same manner as for Compound 1-1 to obtain a white solid, Compound 1-3. ¹H NMR (500 MHz, CDCl₃) δ4.99 (d, J=7.0 Hz, 1H), 4.13 (d, J=5.3 Hz, 1H), 1.59 (s, 2H), 1.43-1.44 (m, 18H, 1.29-1.24 (m, 2H), 1.00 (s, 3H).

The Compound 1-3 (110.5 mg, 0.35 mmol) was dissolved in THF (4 mL) under an ice bath condition, and a 4 mol/L HCl solution was slowly added thereto. A resulting reaction solution was stirred overnight in a dark environment, and then subjected to reduced pressure to remove the THF and the HCl solution to precipitate a white solid, Compound 1-4. The compound was used directly in the next reaction without further purification. ¹H NMR (500 MHz, d6-DMSO) δ8.48 (s, 2H), 3.79 (s, 1H), 1.74-1.62 (m, 2H), 1.48 (ddt, J=85.0, 15.3, 8.2 Hz, 2H), 1.00 (s, 3H).

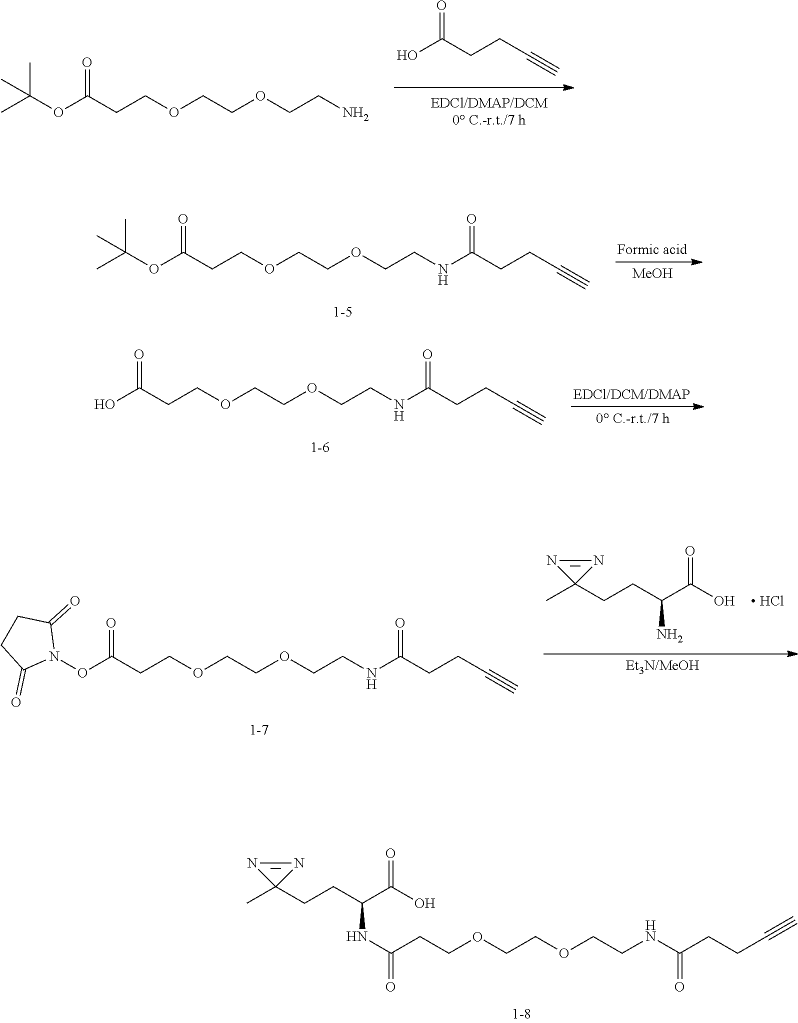

4-Pentynoic acid (119.6 mg, 1.22 mmol), EDCl (216 mg, 1.13 mmol), and 4-dimethylaminopyridine (62.2 mg, 0.51 mmol) were added to a solution of tert-butyl 9-amino-4,7-dioxanonanoate (238 mg, 1.02 mmol) in DCM (5 mL) under an ice bath condition. A resulting reaction solution was heated to room temperature, and subjected to reaction for 7 h. A resulting system was subjected to reduced pressure to remove DCM, extracted with ethyl acetate (3×10 mL), washed with saturated brine (3×10 mL), dried over anhydrous sodium sulfate, and filtered to remove the desiccant. A resulting filtrate was concentrated under reduced pressure. A resulting crude product was purified by silica gel column chromatography (DCM/methanol system) to obtain a yellow oily compound, Compound 1-5 (343.4 mg, 85.5%). ¹H NMR (500 MHz, CDCl₃) δ6.37 (s, 1H), 3.72 (t, J=6.3 Hz, 2H), 3.59 (s, 4H), 3.56-3.53 (m, 2H), 3.45 (q, J=5.2 Hz, 2H), 2.54-2.47 (m, 4H), 2.42 (t, J=7.2 Hz, 2H), 1.98 (t, J=2.6 Hz, 1H), 1.44 (s, 9H).

The Compound 1-5 (216.7 mg, 0.69 mmol) was dissolved in DCM (5 mL) under an ice bath condition, and a formic acid (7 mL) solution was slowly added thereto. A resulting reaction solution was heated to room temperature, stirred for 8 h, and then subjected to reduced pressure to remove the DCM and the formic acid to obtain a Compound 1-6 (170 mg, 96%). The Compound 1-6 was directly used in the next reaction without purification. ¹H NMR (500 MHz, CDCl₃) δ6.54 (s, 1H), 3.76 (t, J=6.1 Hz, 2H), 3.61 (q, J=5.2 Hz, 4H), 3.55 (t, J=5.1 Hz, 2H), 3.45 (q, J=5.3 Hz, 2H), 2.62 (t, J=6.1 Hz, 2H), 2.54-2.49 (m, 2H), 2.43 (t, J=7.1 Hz, 2H), 2.00 (t, J=2.6 Hz, 1H).

The Compound 1-6 (734 mg, 2.85 mmol) was dissolved in a DCM (30 mL) solution under an ice bath condition, and N-hydroxysuccinimide (NHS) (492.2 mg, 4.28 mmol), EDCl (821.8 mg, 4.28 mmol), and 4-dimethylaminopyridine (174.5 mg, 1.43 mmol) were added thereto. A resulting reaction solution was reacted at room temperature overnight, and subjected to post-treatment in the same manner as for Compound 1-5 to obtain a yellow oily compound, Compound 1-7 (863 mg, 82.3%). ¹H NMR (500 MHz, CDCl₃) δ6.35 (s, 1H), 3.85 (t, J=6.1 Hz, 2H), 3.66-3.58 (m, 4H), 3.57-3.52 (m, 2H), 3.45 (q, J=5.2 Hz, 2H), 2.91-2.80 (m, 6H), 2.51 (td, J=7.4, 6.8, 2.5 Hz, 2H), 2.40 (t, J=7.2 Hz, 2H), 1.99 (t, J=2.6 Hz, 1H).

The Compound 1-7 (441.9 mg, 1.25 mmol) and TEA (378.8 mg, 3.75 mmol) were added to a solution of the Compound 1-4 (196 mg, 1.25 mmol) in methanol (4 mL) at room temperature and subjected to reaction in a dark environment for 2 h. A reaction product was subjected to reduced pressure to remove the TEA and the methanol. A resulting residue was dissolved in water, and a pH value thereof was adjusted to 5 with a 1 mol/L HCl solution. A resulting aqueous solution was extracted 3 times with ethyl acetate (3×5 mL). A resulting organic phase was collected, washed with saturated brine, then dried over anhydrous sodium sulfate, and filtered to remove the desiccant. A resulting filtrate was concentrated under reduced pressure. A resulting crude product was purified by silica gel column chromatography (DCM/methanol system) to obtain a pale yellow oily compound, Compound 1-8 (288 mg, yield 58.3%). ¹H NMR (500 MHz, CDCl₃) δ7.03 (d, J=7.7 Hz, 1H), 6.75 (s, 1H), 4.55 (td, J=7.5, 5.6 Hz, 1H), 3.73 (d, J=15.3 Hz, 2H), 3.61 (s, 4H), 3.55 (d, J=6.2 Hz, 2H), 3.49-3.40 (m, 2H), 2.53-2.50 (m, 4H), 2.45 (t, J=6.9 Hz, 2H), 2.02 (t, J=2.6 Hz, 1H), 1.86-1.78 (m, 1H), 1.62-1.55 (m, 1H), 1.51-1.44 (m, 1H), 1.37 (ddd, J=14.6, 11.5, 5.1 Hz, 1H), 1.01 (s, 3H).

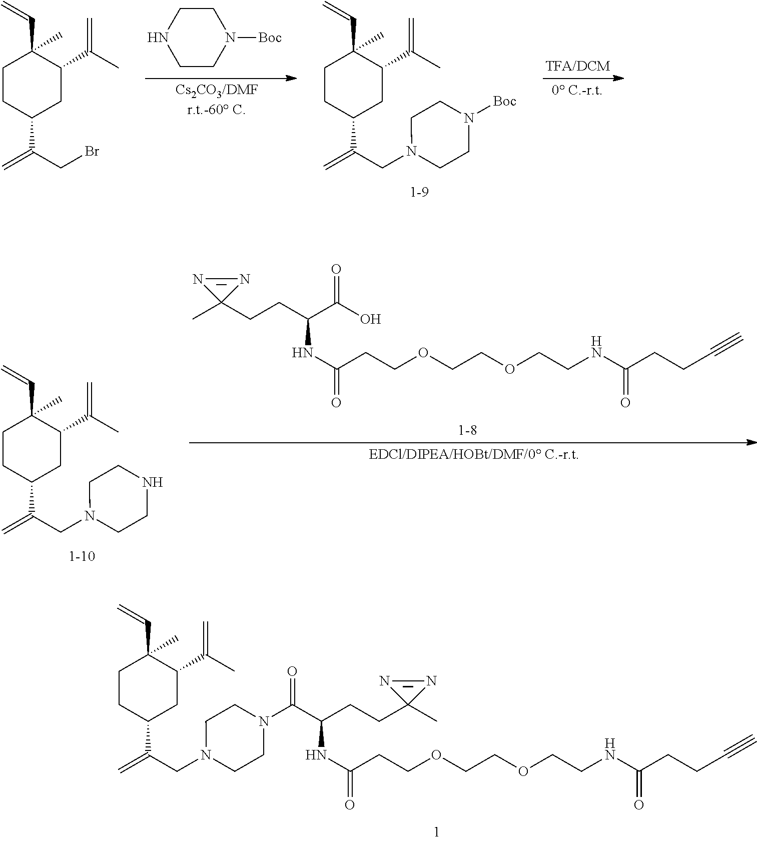 [SMILES: C=C[C@]1(C)CC[C@@H](C(=C)CBr)C[C@H]1C(=C)C ; CC(C)(C)OC(=O)N1CCNCC1 ; C=C[C@]1(C)CC[C@@H](C(=C)CN2CCN(C(=O)OC(C)(C)C)CC2)C[C@H]1C(=C)C ; C=C[C@]1(C)CC[C@@H](C(=C)CN2CCNCC2)C[C@H]1C(=C)C ; C#CCCC(=O)NCCOCCOCCC(=O)N[C@@H](CCC1(C)N=N1)C(=O)O ; C#CCCC(=O)NCCOCCOCCC(=O)N[C@H](CCC1(C)N=N1)C(=O)N1CCN(CC(=C)[C@@H]2CC[C@@](C)(C=C)[C@H](C(=C)C)C2)CC1]

1-Boc-piperazine (153 mg, 0.82 mmol) and vacuum dried cesium carbonate (332 mg, 1.02 mmol) were added to a solution of a 13-brominated compound of elemene (162 mg, 0.68 mmol) in ultra-dry DMF (4 mL) at room temperature. A resulting reaction solution was heated to 60° C., stirred for 4 h, and then subjected to reduced pressure to remove the DMF. A resulting residue was extracted with DCM (3×5 mL). A resulting combined organic phase was washed with water (3×5 mL) and saturated brine (3×5 mL) in sequence, then dried over anhydrous sodium sulfate, and filtered to remove the desiccant. A resulting filtrate was concentrated under reduced pressure. An obtained crude product was purified by silica gel column chromatography (eluted with pure petroleum ether) to obtain a colorless oily compound, Compound 1-9 (189 mg, yield 71.7%). ¹H NMR (500 MHz, CDCl₃) δ5.82 (dd, J=17.3, 11.0 Hz, 1H), 4.91 (m, 4H), 4.83-4.80 (m, 1H), 4.58 (s, 1H), 3.46-3.35 (m, 4H), 2.91 (q, J=13.4 Hz, 2H), 2.32 (s, 4H), 2.08 (m, 1H), 2.02 (dd, J=12.1, 4.0 Hz, 1H), 1.71 (s, 3H), 1.64-1.47 (m, 6H), 1.45 (s, 9H), 1.01 (s, 3H).

The Compound 1-9 (189 mg, 0.488 mmol) was dissolved in dry DCM (1.5 mL). Trifluoroacetic acid (TFA) (1.5 mL) was added thereto at 0° C. A resulting mixture was gradually heated to room temperature and stirred for 3 h. Potassium carbonate (4 mL) and water (5 mL) were slowly added dropwise to quench the reaction. A resulting product was subjected to reduced pressure to remove the TFA and the solvent. A resulting residue was diluted with ethyl acetate (20 mL), and washed with saturated potassium carbonate solution (3 mL). A resulting aqueous layer was back-extracted once (10 mL). A resulting combined organic phase was washed with saturated brine (3 mL), dried over anhydrous sodium sulfate, and filtered to remove the desiccant. A resulting filtrate was concentrated under reduced pressure and then dried to obtain a yellow oily compound, Compound 1-10 (120 mg, yield 85.2%).

The Compound 1-8 (46.3 mg, 0.12 mmol) was dissolved in a DMF (2 mL) solution under an ice bath condition. EDCl (59.4 mg, 0.31 mmol), HOBt (21.6 mg, 0.16 mmol), and DIPEA (46.4 mg, 0.36 mmol) were added thereto. A resulting reaction solution was stirred for 1 h. The Compound 1-10 (45.8 mg, 0.12 mmol) was added thereto. A resulting reaction liquid was reacted in a dark environment at room temperature for 6 h, and subjected to post-treatment in the same manner as for Compound 1-5 to obtain a yellow oily compound, Compound 1 (26.1 mg, 32.6%). ¹H NMR (500 MHz, CDCl₃) δ7.13 (d, J=8.3 Hz, 1H), 7.04 (s, 1H), 5.81 (dd, J=17.3, 11.0 Hz, 1H), 4.98-4.87 (m, 5H), 4.82 (s, 1H), 4.58 (s, 1H), 3.75-3.59 (m, 8H), 3.59-3.49 (m, 6H), 2.93 (q, J=13.4 Hz, 2H), 2.54-2.48 (m, 4H), 2.45-2.33 (m, 6H), 2.06-1.99 (m, 2H), 1.97 (t, J=2.6 Hz, 1H), 1.70 (s, 3H), 1.61-1.34 (m, 10H), 1.00 (d, J=2.6 Hz, 6H).

### Example 2: Preparation of a Compound 2

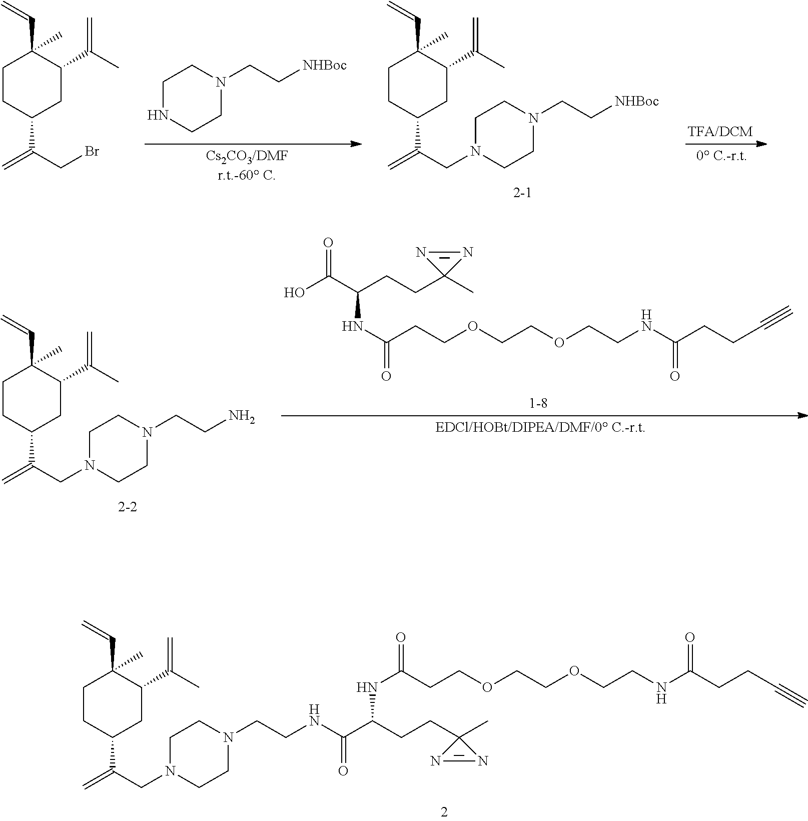

According to the synthesis method in Example 1, a yellow oily compound, Compound 2-1 (537 mg, 76.3%) was obtained in the first step. ¹H NMR (500 MHz, CDCl₃) δ5.82 (dd, J=17.4, 10.9 Hz, 1H), 4.94-4.87 (m, 4H), 4.83-4.80 (m, 1H), 4.58 (s, 1H), 3.22 (d, J=4.6 Hz, 2H), 2.91 (q, J=13.4 Hz, 2H), 2.55-2.29 (m, 10H), 2.10-2.05 (m, 1H), 2.02 (dd, J=12.3, 3.8 Hz, 1H), 1.71 (s, 3H), 1.65-1.47 (m, 6H), 1.44 (s, 9H), 1.01 (s, 3H).

A yellow oily compound, Compound 2-2 (321.7 mg, 78.2%) was obtained in the second step. A yellow oily compound, Compound 2 (119.2 mg, 48.5%) was obtained in the third step. ¹H NMR (500 MHz, CDCl₃) δ7.10 (d, J=8.4 Hz, 2H), 6.71 (s, 1H), 5.81 (dd, J=17.4, 10.9 Hz, 1H), 4.90 (dd, J=17.2, 10.2 Hz, 4H), 4.83-4.78 (m, 1H), 4.57 (s, 1H), 4.41 (q, J=7.2, 6.4 Hz, 1H), 3.74-3.69 (m, 2H), 3.60 (s, 4H), 3.58-3.31 (m, 6H), 2.97-2.87 (m, 2H), 2.54-2.36 (m, 16H), 2.06-2.01 (m, 2H), 1.99-1.97 (m, 1H), 1.70 (s, 3H), 1.64-1.52 (m, 4H), 1.46 (ddd, J=23.4, 13.0, 6.0 Hz, 4H), 1.31-1.27 (m, 2H), 1.01 (d, J=7.6 Hz, 6H).

### Example 3: Preparation of a Compound 3

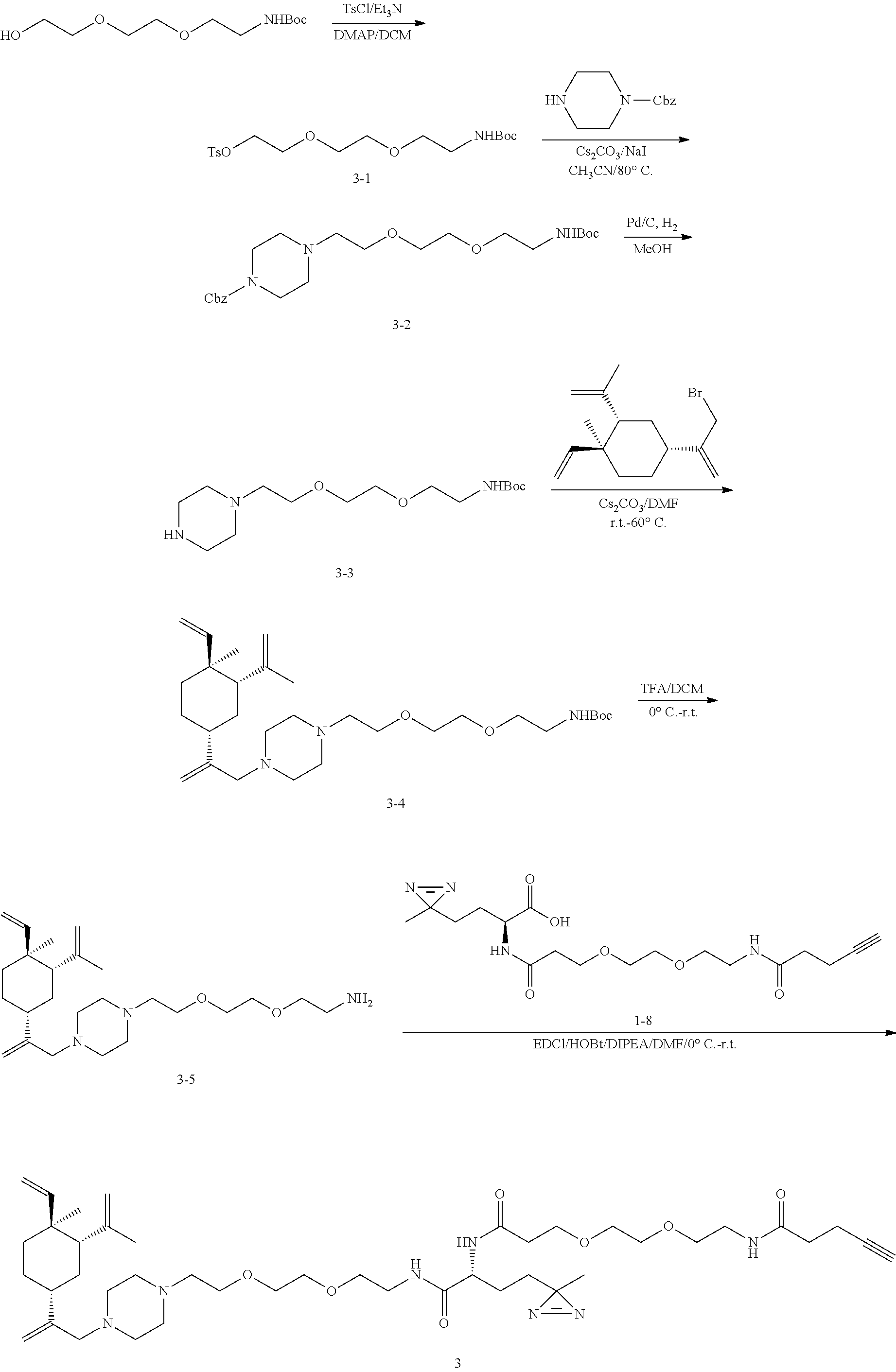

TEA (35.7 mg, 0.354 mmol), p-toluenesulfonyl chloride (40.5 mg, 0.212 mmol), and 4-dimethylaminopyridine (2.12 mg, 0.018 mmol) were added sequentially to a solution of compound 2-[2-(2-T-BOC-aminoethoxy)ethoxy]ethanol (44 mg, 0.177 mmol) in DCM (2 mL) at room temperature. A resulting reaction solution was heated to room temperature and stirred for 7 h. A resulting product was washed with saturated NaCl (3×5 mL), dried over anhydrous Na₂SO₄, and filtered to remove the desiccant, A resulting filtrate was concentrated under reduced pressure. A resulting crude product was purified by silica gel column chromatography (DCM/methanol system) to obtain a colorless oily compound, Compound 3-1 (67.2 mg, yield 94.4%). ¹H NMR (400 MHz, CDCl₃) δ7.79 (d, J=8.3 Hz, 3H), 7.34 (d, J=8.1 Hz, 3H), 4.94 (s, 1H), 4.16-4.17 (m, 2H), 3.68-3.70 (m, 2H), 3.56-3.58 (m, 2H), 3.53-3.55 (m, 2H), 3.48-3.50 (m, 2H), 3.28-3.29 (m, 2H), 2.44 (s, 1H), 1.43 (s, 9H).

Benzyl-1-piperazine carbonate (139.2 mg, 0.633 mmol), vacuum dried cesium carbonate (168 mg, 0.515 mmol), and KI (22 mg, 0.15 mmol) were added to a solution of the Compound 3-1 (166.8 mg, 0.414 mmol) in CH₃CN (2 mL) at room temperature. A resulting reaction solution was heated to 80° C., and stirred for 7 h. A resulting system was then subjected to reduced pressure to remove the CH₃CN. A resulting residual mixture was extracted with DCM (3-5 mL). A resulting combined organic phase was washed with water (3-5 mL) and saturated brine (3×5 mL) in sequence, then dried over anhydrous sodium sulfate, and filtered to remove the desiccant. A resulting filtrate was concentrated under reduced pressure. A resulting crude product was purified by silica gel column chromatography (DCM/methanol system) to obtain a colorless oily compound, Compound 3-2 (508 mg, yield 45.1%). ¹H NMR (400 MHz, CDCl₃) δ7.34 (d, J=3.9 Hz, 5H), 5.29 (s, 1H), 5.12 (s, 2H), 3.60 (d, J=5.6 Hz, 6H), 3.54-3.49 (m, 6H), 3.30 (q, J=4.9 Hz, 2H), 2.60 (t, J=5.7 Hz, 2H), 2.46 (s, 4H), 1.43 (s, 9H).

10% Pd/C (19.2 mg) was added to a solution of the Compound 3-2 (59.3 mg, 0.132 mmol) in MeOH (2 mL) at room temperature, and a resulting reaction solution was reacted overnight under H₂ atmosphere. A resulting product was filtered to remove the 10% Pd/C. A resulting filtrate was concentrated under reduced pressure to obtain a Compound 3-3 (35 mg, 84%), The Compound 3-3 was directly used in the next step without column chromatography.

Referring to the synthesis method of Compound 1-9 in Example 1, a reaction was conducted to obtain a Compound 3-4 (191.7 mg, yield 70.2%) as a colorless oil. ¹H NMR (500 MHz, CDCl₃) δ5.82 (dd, J=17.4, 10.8 Hz, 1H), 4.93-4.87 (m, 4H), 4.82-4.79 (m, 1H), 4.58 (s, 1H), 3.60 (d, J=6.9 Hz, 6H), 3.53 (t, J=5.0 Hz, 2H), 3.30 (t, J=5.4 Hz, 2H), 2.95-2.86 (m, 2H), 2.62-2.40 (m, 8H), 2.01 (dd, J=12.1, 3.9 Hz, 2H), 1.70 (s, 3H), 1.66-1.45 (m, 6H), 1.44 (s, 9H), 1.00 (s, 3H).

Referring to the synthesis method of Compound 1-10 in Example 1, a reaction was conducted to obtain a Compound 3-5 (132.9 mg, yield 85.9%) as a yellow oily compound.

Referring to the synthesis method of Compound 1 in Example 1, a reaction was conducted to obtain a Compound 3 (89.3 mg, yield 35.6%) as a yellow oily compound. ¹H NMR (500 MHz, CDCl₃) δ7.66 (s, 1H), 7.36 (d, J=7.3 Hz, 1H), 7.14 (s, 1H), 5.82 (dd, J=17.8, 10.5 Hz, 1H), 5.05-4.95 (m, 2H), 4.92 (d, J=4.5 Hz, 1H), 4.89 (s, 1H), 4.84-4.81 (m, 1H), 4.58 (s, 1H), 4.17-4.10 (m, 1H), 3.76-3.68 (m, 3H), 3.63-3.59 (m, 8H), 3.58-3.55 (m, 4H), 3.52 (dq, J=6.6, 3.2, 2.7 Hz, 2H), 3.41 (dd, J=11.0, 7.8 Hz, 3H), 3.05 (s, 2H), 2.84 (s, 6H), 2.53 (td, J=7.3, 6.7, 3.7 Hz, 4H), 2.46 (t, J=7.0 Hz, 2H), 2.13 (d, J=22.8 Hz, 4H), 2.07 (s, 1H), 2.05 (s, 1H), 1.99 (d, J=2.5 Hz, 1H), 1.71 (s, 3H), 1.59-1.39 (m, 10H), 1.01 (d, J=8.0 Hz, 6H).

### Example 4: Preparation of a Compound 4

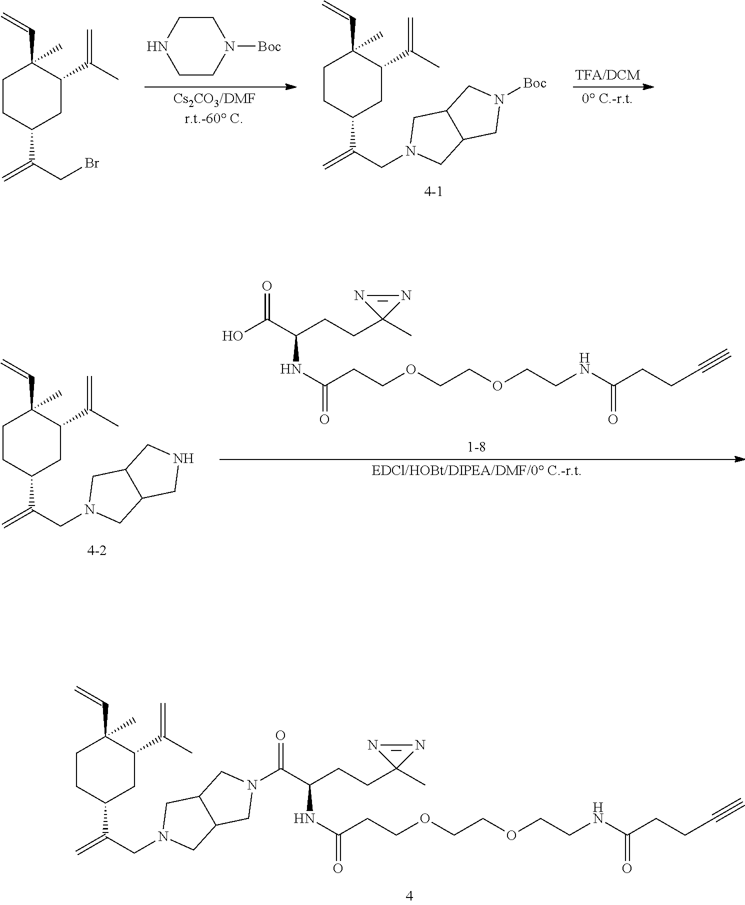 [SMILES: C=C[C@]1(C)CC[C@@H](C(=C)CBr)C[C@H]1C(=C)C ; CC(C)(C)OC(=O)N1CCNCC1 ; C=C[C@]1(C)CC[C@@H](C(=C)CN2CC3CN(C(=O)OC(C)(C)C)CC3C2)C[C@H]1C(=C)C ; C=C[C@]1(C)CC[C@@H](C(=C)CN2CC3CNCC3C2)C[C@H]1C(=C)C ; C#CCCC(=O)NCCOCCOCCC(=O)N[C@H](CCC1(C)N=N1)C(=O)O ; C#CCCC(=O)NCCOCCOCCC(=O)N[C@H](CCC1(C)N=N1)C(=O)N1CC2CN(CC(=C)[C@@H]3CC[C@@](C)(C=C)[C@H](C(=C)C)C3)CC2C1]

Referring to the synthesis method of Compound 1-9 in Example 1, a reaction was conducted to obtain a Compound 4-1 (113.87 mg, yield 67.6%) as a yellow oily compound. ¹H NMR (500 MHz, CDCl₃) δ5.82 (dd, J=17.3, 11.0 Hz, 1H), 4.93 (s, 1H), 4.90 (dd, J=7.6, 1.3 Hz, 1H), 4.88 (s, 1H), 4.85 (s, 1H), 4.82-4.80 (m, 1H), 4.58 (s, 1H), 3.55 (s, 2H), 3.19 (s, 2H), 2.99 (q, J=15 Hz, 2H), 2.76 (s, 2H), 2.54 (s, 2H), 2.39 (d, J=7.1 Hz, 2H), 2.10-1.99 (m, 2H), 1.70 (s, 3H), 1.67-1.48 (m, 6H), 1.45 (s, 9H), 1.00 (s, 3H).

Referring to the synthesis method of Compound 1-10 in Example 1, a reaction was conducted to obtain a Compound 4-2 (77.6 mg, yield 90%) as a yellow oily compound.

Referring to the synthesis method of Compound 1 in Example 1, a reaction was conducted to obtain a Compound 4 (38.3 mg, yield 32.6%) as a yellow oily compound. LCMS: m/z 693.5[M+H]⁺. ¹H NMR (500 MHz, CDCl₃) δ7.17 (ddd, J=45.8, 12.9, 6.9 Hz, 2H), 5.82 (ddd, J=17.4, 10.9, 3.5 Hz, 1H), 4.97-4.86 (m, 4H), 4.82 (dt, J=5.0, 1.8 Hz, 1H), 4.74 (td, J=7.9, 4.6 Hz, 1H), 4.58 (dd, J=4.6, 2.0 Hz, 1H), 3.71 (q, J=5.9 Hz, 4H), 3.65-3.51 (m, 10H), 3.08-2.95 (m, 2H), 2.63-2.45 (m, 12H), 2.05-1.99 (m, 3H), 1.98 (t, J=2.6 Hz, 1H), 1.71 (d, J=4.2 Hz, 3H), 1.61-1.35 (m, 12H), 1.02-0.98 (m, 6H).

### Example 5: Preparation of a Compound 5

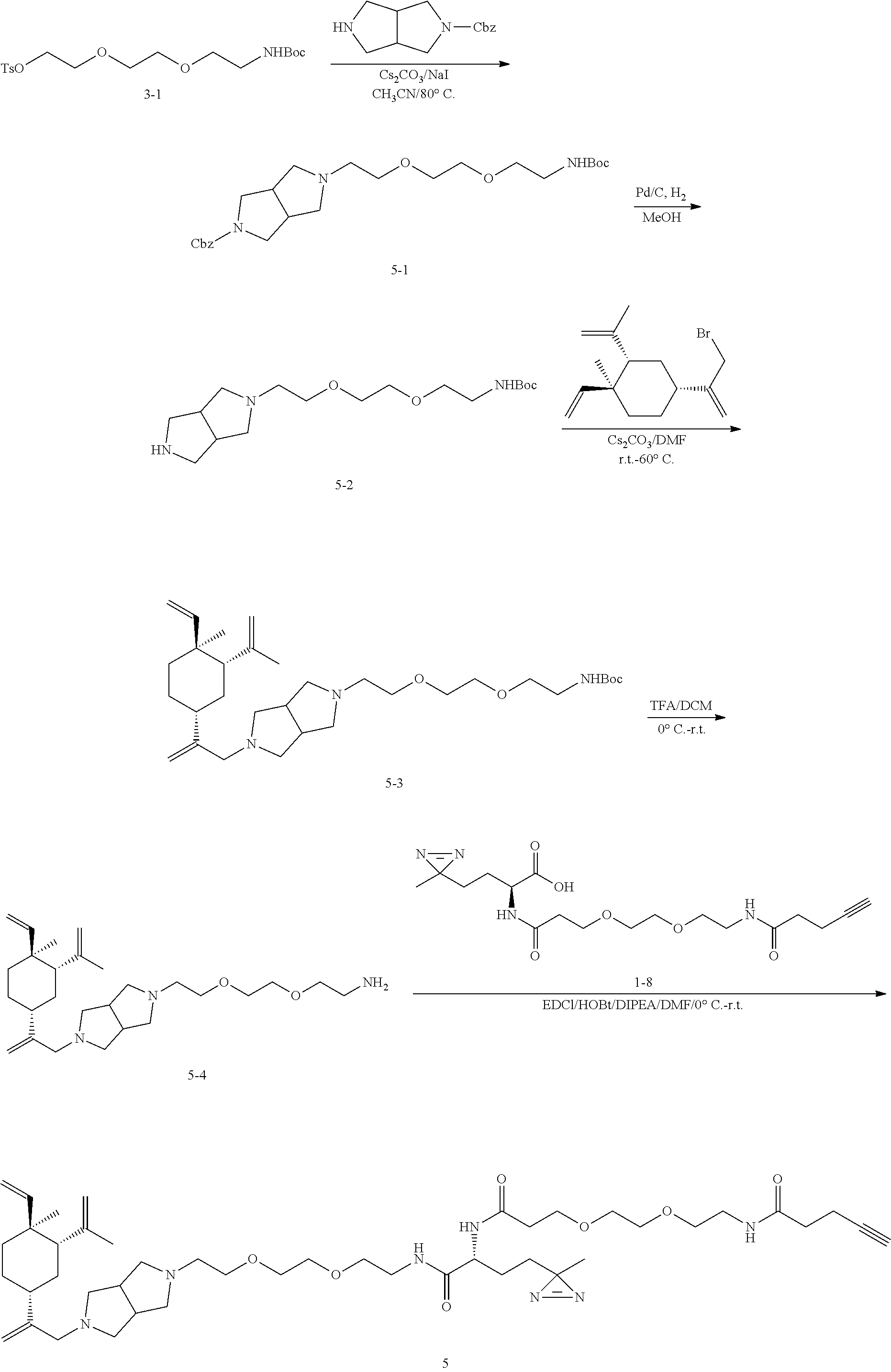

Referring to the synthesis method of Compound 3-2 in Example 3, a reaction was conducted to obtain a Compound 5-1 (56.8 mg, yield 45.1%). ¹H NMR (500 MHz, CDCl₃) δ7.36-7.27 (m, 5H), 5.11 (s, 3H), 3.59 (s, 6H), 3.57 (d, J=5.9 Hz, 2H), 3.52 (t, J=5.1 Hz, 2H), 3.40-3.26 (m, 4H), 2.82 (s, 4H), 2.65 (t, J=5.9 Hz, 2H), 2.36 (s, 2H), 1.43 (s, 9H).

Referring to the synthesis method of Compound 3-3 in Example 3, a reaction was conducted to obtain a Compound 5-2. The Compound 5-2 was directly used in the next step without column chromatography.

Referring to the synthesis method of Compound 1-9 in Example 1, a reaction was conducted to obtain a Compound 5-3 (240 mg, yield 72.8%). ¹H NMR (500 MHz, CDCl₃) δ5.82 (dd, J=17.4, 10.9 Hz, 1H), 4.94-4.87 (m, 4H), 4.83-4.80 (m, 1H), 4.58 (s, 1H), 3.22 (d, J=4.6 Hz, 2H), 2.91 (q, J=13.4 Hz, 2H), 2.55-2.29 (m, 10H), 2.10-2.05 (m, 1H), 2.02 (dd, J=12.3, 3.8 Hz, 1H), 1.71 (s, 3H), 1.65-1.47 (m, 6H), 1.44 (s, 9H), 1.01 (s, 3H).

Referring to the synthesis method of Compound 1-10 in Example 1, a reaction was conducted to obtain a Compound 5-4 (188.1 mg, yield 96%) as a yellow oily compound.

Referring to the synthesis method of Compound 1 in Example 1, a reaction was conducted to obtain a Compound 5 (57.5 mg, yield 30.2%) as a yellow oily compound. ¹H NMR (500 MHz, CDCl₃) δ7.25 (d, J=7.6 Hz, 2H), 7.08 (s, 1H), 5.81 (dd, J=17.4, 10.9 Hz, 1H), 4.96-4.85 (m, 4H), 4.83-4.78 (m, 1H), 4.58 (s, 1H), 4.54-4.42 (m, 1H), 3.72 (dq, J=8.7, 4.7, 4.2 Hz, 4H), 3.64-3.51 (m, 13H), 3.47-3.36 (m, 3H), 3.07-3.00 (m, 2H), 2.84 (s, 5H), 2.62-2.39 (m, 9H), 2.38-2.19 (m, 4H), 2.00-1.98 (m, 1H), 1.70 (s, 3H), 1.63-1.42 (m, 8H), 1.28 (d, J=5.5 Hz, 4H), 1.00 (s, 6H).

### Example 6: Preparation of a Compound 6

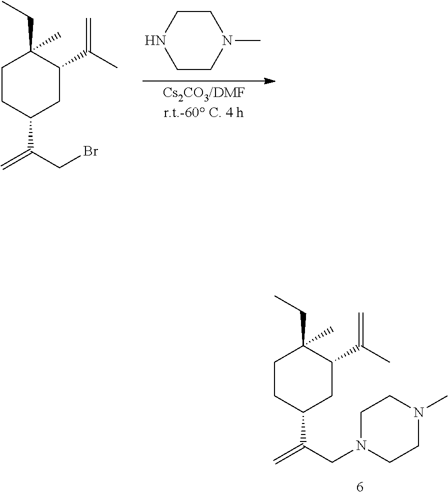

Referring to the synthesis method of Compound 1-9 in Example 1, a reaction was conducted to obtain a Compound 6 (113.6 mg, yield 62.8%) as a yellow oily compound. ¹H NMR (500 MHz, CDCl₃) δ5.82 (dd, J=17.5, 10.91 Hz, 1H), 4.94-4.87 (m, 4H), 4.81 (p, J=1.6 Hz, 1H), 4.58 (d, J=2.0 Hz, 1H), 2.96-2.86 (m, 2H), 2.41 (s, 8H), 2.28 (s, 3H), 2.01 (dd, J=12.3, 3.9 Hz, 2H), 1.70 (d, J=2.3 Hz, 3H), 1.64-1.40 (m, 6H), 1.00 (s, 3H).

### Example 7: Preparation of a Compound 7

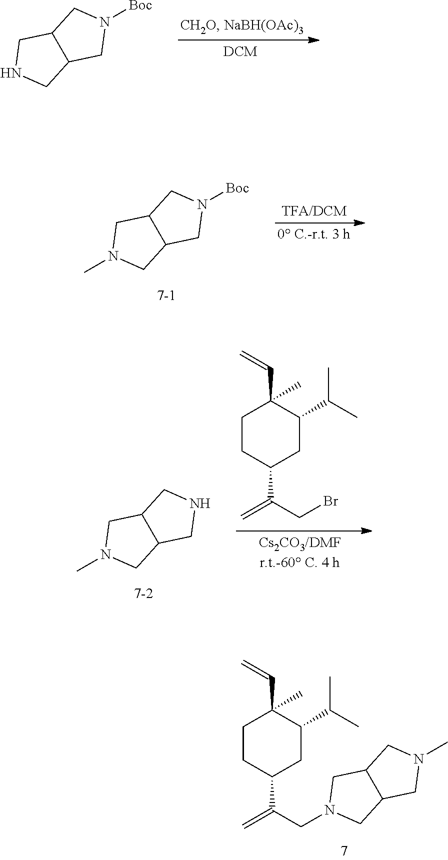

Sodium acetoxyborohydride (1.5 g, 7.1 mmol) and a methanol solution (355 mg, 11.8 mmol) were added to a solution of cis-2-BOC-hexahydropyrrolo[3,4-c]pyrrole compound (212.3 mg, 2.35 mmol) in DCM (10 mL) at room temperature. A resulting reaction solution was reacted overnight at room temperature. The K₂CO₃ was added thereto to quench the reaction. A pH value of a resulting product was adjusted to about 9, and the adjusted product was subjected to post-treatment in the same manner as for Compound 1-1 to obtain a colorless oily compound, Compound 7-1 (523 mg, yield 97.6%). ¹H NMR (500 MHz, CDCl₃) δ3.53 (s, 2H), 3.22 (s, 2H), 2.80 (dh, J=9.3, 4.0 Hz, 2H), 2.65-2.59 (m, 2H), 2.36 (d, J=9.0 Hz, 2H), 2.31 (s, 3H), 1.44 (s, 9H).

Referring to the synthesis of Compound 1-10 in Example 1, a reaction was conducted to obtain a Compound 7-2 (103 mg, yield 92%).

Referring to the synthesis of Compound 1-9 in Example 1, a reaction was conducted to obtain a Compound 7 (56.3 mg, yield 53.6%) as a yellow oily compound. ¹H NMR (500 MHz, CDCl₃) δ5.85-5.76 (m, 1H), 5.05-4.77 (m, 5H), 4.58 (d, J=2.1 Hz, 1H), 3.17-3.04 (m, 3H), 2.97-2.87 (m, 2H), 2.59-2.38 (m, 8H), 2.09-1.96 (m, 2H), 1.71 (d, J=2.0 Hz, 3H), 1.66-1.17 (m, 8H), 1.00 (s, 3H).

### Example 8: Preparation of a Compound 8

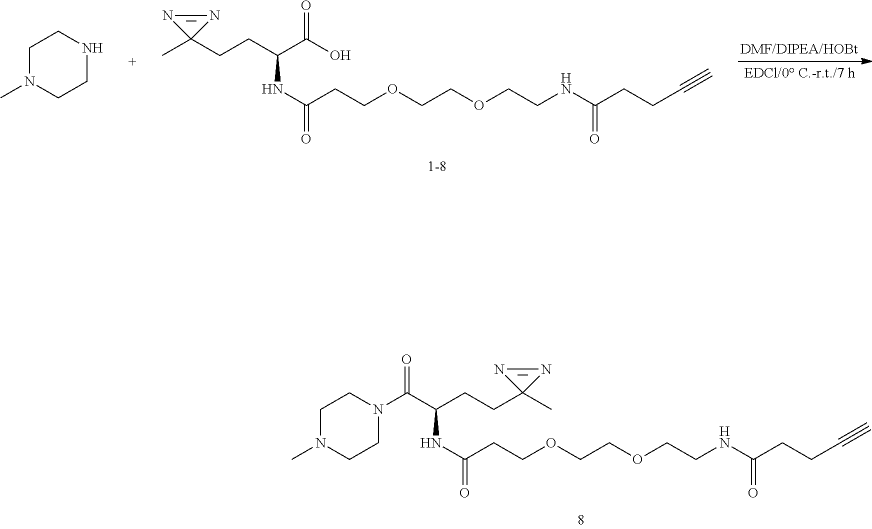

Referring to the synthesis method of Compound 1 in Example 1, a reaction was conducted to obtain a Compound 8 (57.5 mg, yield 40.6%) as a yellow oily compound. ¹H NMR (400 MHz, Chloroform-d) δ4.90 (td, J=7.8, 4.4 Hz, 1H), 3.74-3.56 (m, 14H), 2.46-2.37 (m, 8H), 2.33 (s, 5H), 1.98 (t, J=2.6 Hz, 1H), 1.67 (s, 2H), 1.25 (s, 2H), 1.01 (s, 3H).

### Example 9: Evaluation of In Vitro Antitumor Activity (Tumor Cell Proliferation Inhibition Experiment)

### 1. Experimental Equipment and Reagents

### 1.1 Instruments

Biosafety cabinet (Shanghai Bio-Gene Biotechnology Co., Ltd.), constant-temperature carbon dioxide incubator (THERMO), enzyme-linked immunoassay analyzer (Spark), inverted microscope (Nikon), a set of pipette guns (Eppendorf), and centrifuge (Beckman Coulter).

### 1.2 Reagents

DMEM (BI), RPMI 1640 (BI), Fatal Bovine Serum (Excell), PBS (Zhejiang Cienry Biotechnology Co., Ltd.), Trypsin (Zhejiang Cienry Biotechnology Co., Ltd.), DMSO (Coolaber), CCK-8 (Coolaber), Double-antibody (Gbico).

### 1.3 Cell Lines

Human liver cancer cell Huh1, human lung cancer cell A549, and human brain glioma cell U87-MG.

### 2. Experimental Methods

### 2.1 Cell Resuscitation and Routine Culture

(1) A cell laboratory was routinely disinfected and a clean bench was subjected to ultraviolet radiation for more than 30 min.
(2) A culture solution (DMEM) and 0.25% trypsin were preheated in a constant-temperature water bath at 37° C. for 20 min, and dimethyl sulfoxide (DMSO) was refrigerated in a refrigerator at 4° C. for 30 min.
(3) A cryopreserved tube was taken out of a liquid nitrogen storage tank, immediately placed in a 37° C. water bath and shaken rapidly until s cryopreserved liquid was completely thawed.
(4) A cell suspension was transferred into a centrifuge tube, the culture solution was slowly added thereto, and the centrifuge tube was transferred into a centrifuge for centrifugation (120×g, 5 min).
(5) A cell concentration was adjusted with the culture solution, and resulting adjusted cells were shaken well, and placed in a 37° C., 5% CO₂ incubator for culture.
(6) After the cells had attached for 24 h, the culture solution thereof was changed. When a cell density reached 80% to 90%, the cells were digested with trypsin and then subcultured.

### 2.2. Experiment Method of Tumor Cell Proliferation Inhibition

(1) A A549 cell suspension was adjusted to 5×10⁴ cells/mL using a DMEM culture medium. 100 μL of an adjusted cell suspension was added to each well of a 96-well cell culture plate, such that the 96-well cell culture plate had a final cell concentration of 5,000 cells/well, and cultured in a 37° C., 5% CO₂ incubator for 24 h.
(2) An elemene bulk drug was diluted to a required concentration using a complete culture medium, the culture medium was removed from the wells, 100 μL of the drug-containing culture medium was added to each well, and 3 replicate wells were set up for each drug concentration, while a control group and a blank group were set up.
(3) The 96-well plate was incubated in a 37° C., 5% CO₂ incubator for 48 h.
(4) After 48 h of drug treatment, the culture medium was sucked out and 100 μL of a culture medium containing 10% CCK-8 was added to each well. The cells were cultured in the incubator for 1 h and an absorbance at 450 nm was measured using a THERMO FISHER Multiskan FC microplate reader.
(5) IBM SPSS Statistics 22.0 software was used to plot an S-shaped dose-survival curve using a non-linear regression model, and a survival rate and an inhibition rate were calculated using the following formulas:

$$Cell viability = [ ( As - Ab ) / ( Ac - Ab ) ] \times 100 \%$$

$$Inhibition rate = [ ( Ac - As ) / ( Ac - Ab ) ] \times 100 \%$$

As: absorbance of the experimental well (culture medium containing cells, CCK-8, and the drug to be tested)
Ac: absorbance of the control well (culture medium containing cells, CCK-8, and no drug to be tested)
Ab: absorbance of the blank wells (culture medium without cells and drug to be tested, CCK-8)

### 3 Experimental Results

According to the above experimental method, CCK-8 was used to determine the proliferation inhibitory effects of the isolated and purified β-elemene monomer, elemene photoaffinity probe and control compounds on three human cancer cells, namely A549 human non-small cell lung cancer cells, U87-MG human malignant glioma cells, and Huh1 human liver cancer cells. The results are shown in Table 1.

TABLE 1
IC₅₀ of β-elemene, β-elemene photoaffinity probes (Compounds
1 to 5) and control compounds (Compounds 6 to 8)

| | IC₅₀ (μmol/mL) | | |
|---|---|---|---|
| Compound | A549 | U87 | Huh1 |
| β-elemene | 212.80 ± 5.29 | 217.70 ± 9.58 | 305.50 ± 31.68 |
| 1 | 29.34 ± 1.63 | 38.72 ± 1.91 | 18.71 ± 1.541 |
| 2 | 37.75 ± 3.36 | 25.52 ± 4.79 | 20.12 ± 0.16 |
| 3 | 44.63 ± 7.68 | 19.32 ± 3.30 | 22.82 ± 3.07 |
| 4 | 48.35 ± 1.70 | 47.17 ± 2.21 | 54.50 ± 3.26 |
| 5 | 28.37 ± 3.36 | 14.06 ± 1.72 | 17.09 ± 0.80 |
| 6 | 102.60 ± 7.47 | 73.27 ± 8.27 | 92.34 ± 10.13 |
| 7 | 43.43 ± 2.14 | 23.26 ± 0.84 | 24.33 ± 4.02 |
| 8 | ND | ND | ND |

NOTE:
ND meant no anti-tumor activity was detected.

The results of in vitro antitumor activity results in Table 1 show that the inhibitory activities of the 7 compounds tested on human A549 lung cancer cells, human Huh1 liver cancer cells, and human U87-MG brain glioma cells are stronger than those of β-elemene. For human A549 lung cancer cells, the IC₅₀ values of 3 compounds (1, 2, 5) are more than 5 times lower than those of β-elemene; for human Huh1 liver cancer cells, the IC₅₀ values of 5 compounds (1, 2, 3, 5, 7) are more than 10 times lower than those of β-elemene; for human U87-MG malignant glioma cells, the IC₅₀ values of 2 compounds (3, 5) are more than 10 times lower than those of β-elemene. The results show that the in vitro antitumor activity of β-elemene is enhanced by introducing polar groups containing oxygen atom and nitrogen atom and PEG chains with enhanced water solubility while ensuring the integrity of the β-elemene skeleton.

### Example 10: Pre-Targeted Imaging Studies Using Molecular Probe in Living Cells

In order to determine whether the molecular probe could enter the cell and explore the binding site of the targeted biomacromolecule thereof, the experiment was conducted as follows.

### 1. Experimental Equipment and Reagents

### 1.1 Instruments

Portable LED UV flaw detection lamp (LUYOR), laser confocal microscope (Olympus, FV3000RS), constant-temperature carbon dioxide incubator (THERMO)

### 1.2 Reagents

DMEM culture medium, PBS, 4% paraformaldehyde tissue cell fixative, Triton X-100, 5% BSA blocking solution, DAPI staining solution, Cu₂SO₄, sodium ascorbate, THPTA, N₃-TAMRA

### 2. Experimental Methods

(1) 10⁵ Huh1 liver cancer cells were inoculated into glass-bottomed culture dishes and cultured in a 37° C., 5% CO₂ incubator until the cells adhered to wall.
(2) DMEM culture medias containing different drug concentrations were added to the culture dishes and incubated overnight in the 37° C., 5% CO₂ incubator.
(3) After removing the drug-containing culture mediums, resulting cells were washed with pre-cooled PBS (3-1 mL) for 3 min, placed on ice, and irradiated at 365 nm for 15 min.
(4) Resulting irradiated cells were treated with 1 mL of 4% paraformaldehyde tissue cell fixative for 15 min, the paraformaldehyde tissue cell fixative was removed, and resulting cells were washed with pre-cooled PBS.
(5) Resulting washed cells were permeabilized with 500 μL of 0.1% Triton X-100 in PBS for 10 min, then blocked with 1 mL of 5% BSA blocking solution for 30 min, the blocking solution was removed, and resulting cells were washed with pre-cooled PBS (3×1 mL).
(6) 200 μL of freshly prepared Click reaction solution (20 μM N₃-TAMRA, 400 μM Cu₂SO₄, 400 μM THPTA, 2 mM sodium ascorbate) was added to washed cells obtained in step
(5), incubated under shaking at room temperature for 2 h, the reaction solution was removed, and resulting cells were washed with pre-cooled PBS (3×1 mL) for 3 min.
(7) 200 μL of DAPI staining solution was added to washed cells obtained in step (6) and stained for 6 min, the staining solution was removed, and resulting cells were washed with pre-cooled PBS (3×1 mL) for 3 min, and then imaged with a laser scanning confocal microscope.

### 3 Experimental Results

According to the above experimental method, the cell images of the β-elemene photoaffinity probe (Compound 2) and a control Compound 6 and β-elemene were measured. The results are shown in FIGURE.

The results show that with the addition of DMSO or Compound 2 (20 μM) and without UV irradiation, there is no intracellular Rhodamine Red fluorescence; with the addition of Compound 2 (20 μM) and with UV irradiation, there is strong intracellular Rhodamine Red fluorescence; with the addition of Compound 2 (20 μM) and Compound 6 (20 PM), as well as Compound 2 (20 μM) and β-elemene (50 μM) and with UV irradiation, the intensity the intracellular Rhodamine Red fluorescence is greatly weakened. The above results indicate that the binding of the β-elemene photoaffinity probe (Compound 2) to the target is non-covalent; both Compound 6 and β-elemene can competitively bind to Compound 2.

The above descriptions are merely preferred embodiments of the present disclosure. It should be noted that a person of ordinary skill in the art may further make several improvements and modifications without departing from the principle of the present disclosure, but such improvements and modifications should be deemed as falling within the scope of the present disclosure.

1. A β-elemene derivative containing a photoaffinity group diazirine, or an optical isomer, a racemate, a single enantiomer, and a possible diastereomer thereof, or a pharmaceutically acceptable salt, a prodrug, a deuterated derivative, a hydrate, and a solvate thereof, wherein the β-elemene derivative containing the photoaffinity group diazirine has a structure shown in Formula (I): 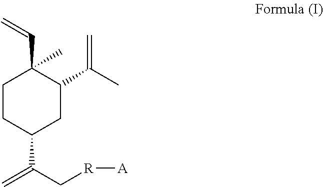
wherein
A represents 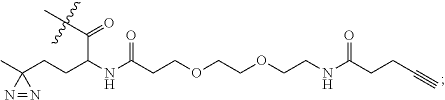 [SMILES: *C(=O)C(CCC1(C)N=N1)NC(=O)CCOCCOCCNC(=O)CCC#C]
wherein “ [symbol] ” represents a site where A is linked to R; and
R is independently selected from the group consisting of the following structural fragments: 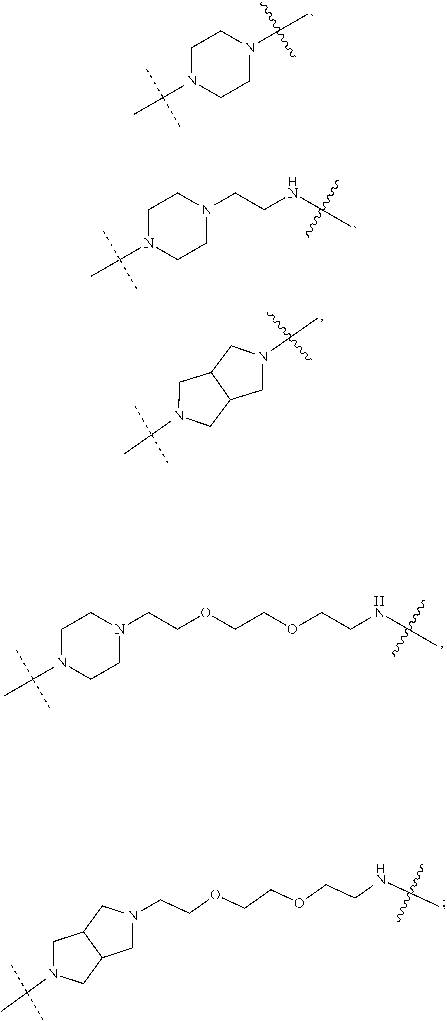
wherein “ [symbol] ” represents a site where R is linked to elemene, and “ [symbol] ” represents a site where R is linked to A.

2. The β-elemene derivative containing the photoaffinity group diazirine of claim 1, wherein the β-elemene derivative containing the photoaffinity group diazirine has the structure being any one selected from the group consisting of Formula 1 to Formula 5:

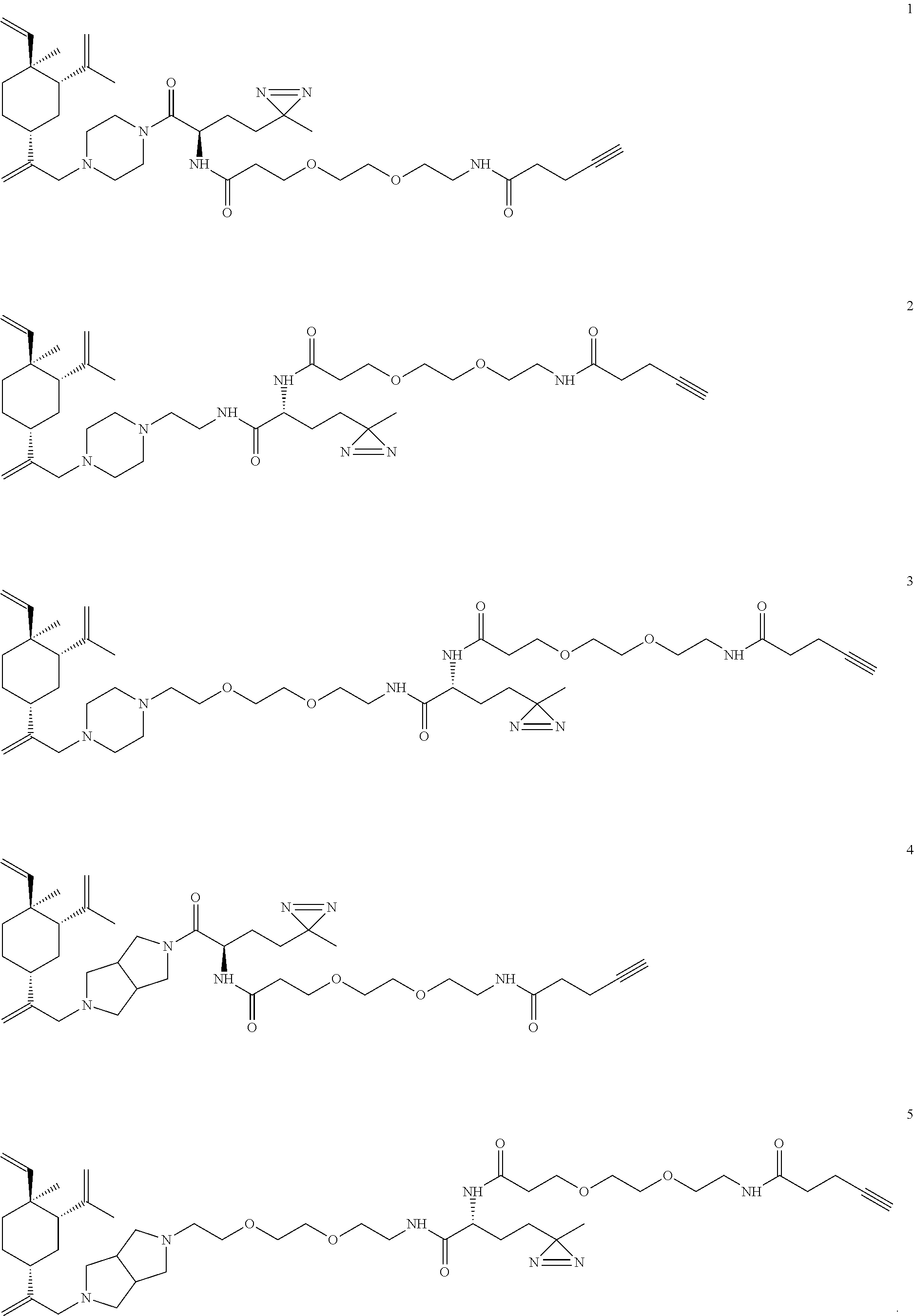

3. A method for preparing the β-elemene derivative containing the photoaffinity group diazirine of claim 1, comprising:
(1) subjecting a β-elemene A-1 to bromine substitution at a 13-allyl position to obtain an intermediate A-2;
(2) linking an R structural fragment A-3 containing a nitrogen heteroatom functional group to the intermediate A-2 through selective nucleophilic substitution to obtain an intermediate A-4;
(3) subjecting a structural fragment at a 13-β-elemene position of the intermediate A-4 to deprotection to obtain an intermediate A-5; and
(4) subjecting the intermediate A-5 and a probe fragment A-OH to intermolecular amide condensation to obtain the β-elemene derivative containing the photoaffinity group diazirine having the structure shown in Formula (I);
wherein a synthetic path is as follows:

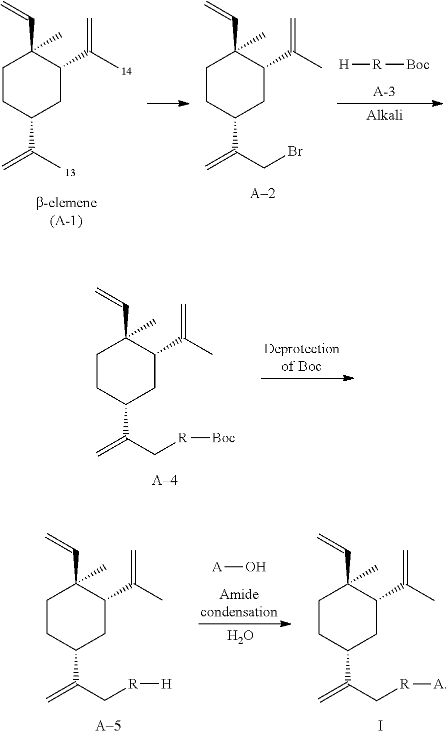

4. A method for inhibiting a proliferation activity of a tumor cell line, comprising:
using the β-elemene derivative containing the photoaffinity group diazirine of claim 1, or the optical isomer, the racemate, the single enantiomer, and the possible diastereomer thereof, or the pharmaceutically acceptable salt, the prodrug, the deuterated derivative, the hydrate, and the solvate thereof as a β-elemene photoaffinity molecular probe.

5. (canceled)

6. The method of claim 4, wherein the tumor cell line is one selected from the group consisting of a human liver cancer cell Huh1, a human lung cancer cell A549, and a human brain glioma cell U87-MG.

7. A method for screening a β-elemene interacting protein, comprising:
using the β-elemene derivative containing the photoaffinity group diazirine of claim 1, or the optical isomer, the racemate, the single enantiomer, and the possible diastereomer thereof, or the pharmaceutically acceptable salt, the prodrug, the deuterated derivative, the hydrate, and the solvate thereof as a photoaffinity molecular probe.

8. The method for screening the β-elemene interacting protein of claim 7, wherein the photoaffinity molecular probe covalently binds to a protein in a cytoplasmic after being irradiated with a wavelength of 365 nm.

9. (canceled)

10. The method of claim 3, wherein the β-elemene derivative containing the photoaffinity group diazirine has the structure being any one selected from the group consisting of Formula 1 to Formula 5:

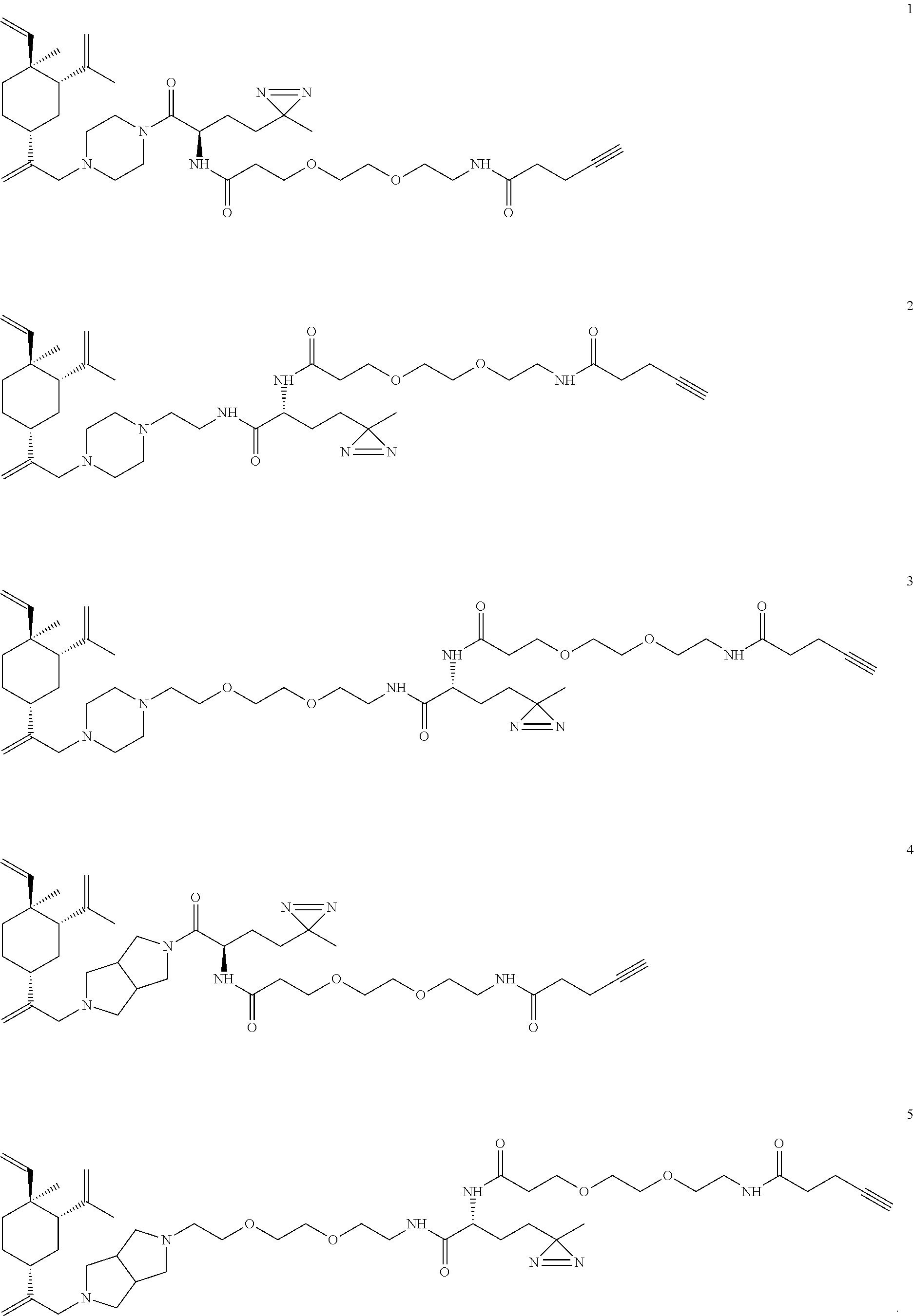

11. The method of claim 4, wherein the β-elemene derivative containing the photoaffinity group diazirine has the structure being any one selected from the group consisting of Formula 1 to Formula 5:

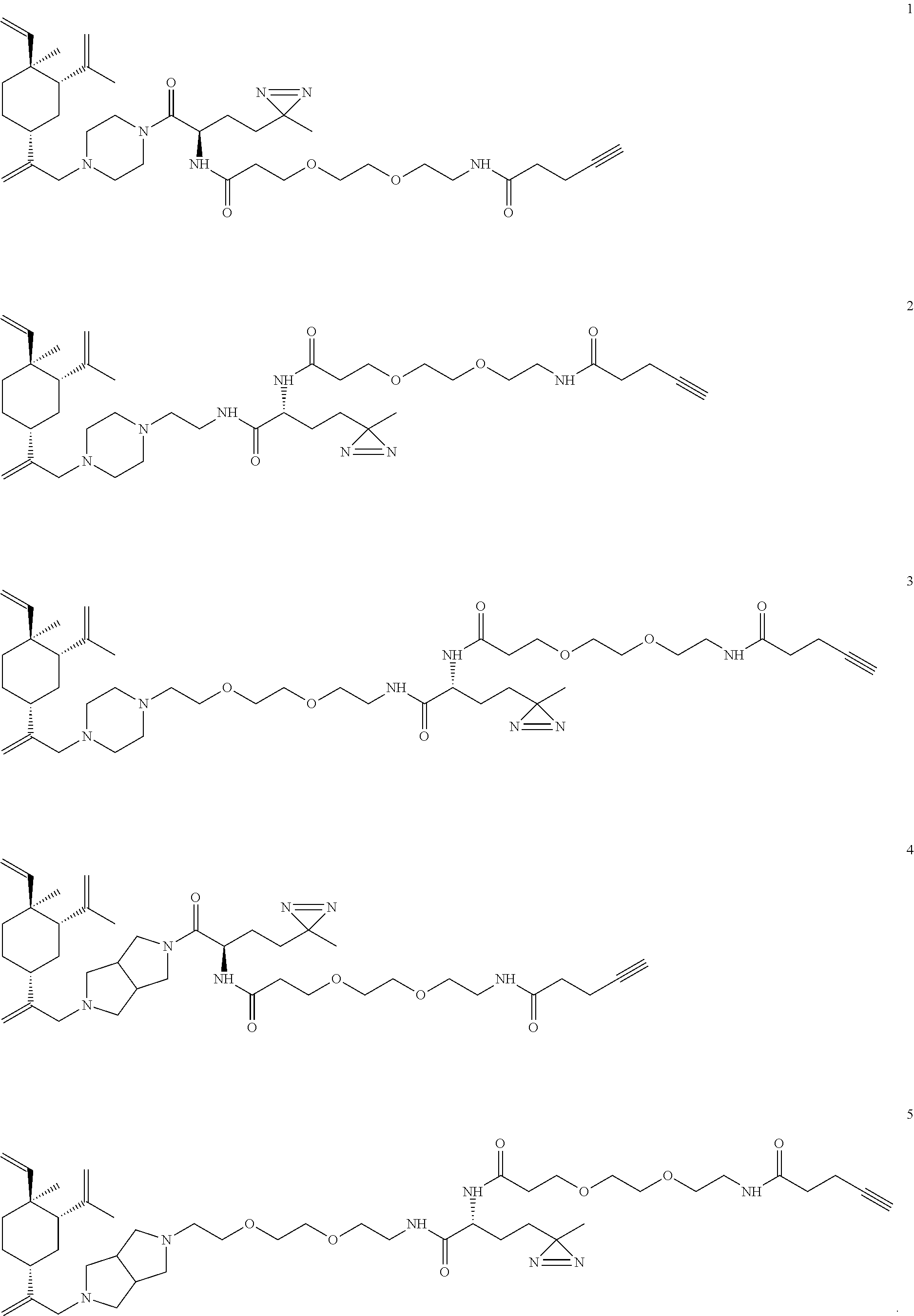

12. The method of claim 7, wherein the β-elemene derivative containing the photoaffinity group diazirine has the structure being any one selected from the group consisting of Formula 1 to Formula 5: [chemical structure]
